(19) United States
(12) Patent Application Publication
(10) Pub. No.: US 2025/0319045 A1
(43) Pub. Date: Oct. 16, 2025

(54) TRANSETHOSOME-ENCAPSULATED AGENTS AND METHODS OF USE THEREOF

(71) Applicant: Northwestern University, Evanston, IL (US)

(72) Inventors: Oukseub Lee, Evanston, IL (US); Seema Khan, Evanston, IL (US)

(21) Appl. No.: 19/174,036

(22) Filed: Apr. 9, 2025

Related U.S. Application Data
(60) Provisional application No. 63/632,256, filed on Apr. 10, 2024.

(51) Int. Cl. A61K 31/138; A61K 9/00; A61K 9/1272; A61K 31/573; A61K 47/10; A61K 47/28

(52) U.S. Cl. CPC A61K 31/138; A61K 9/0014; A61K 9/1272; A61K 31/573; A61K 47/10; A61K 47/28

(57) ABSTRACT

Provided herein are transethosome-encapsulated endocrine agents and methods of use thereof, such as for treatment of cancer.

### PRIORITY STATEMENT

This application claims priority to U.S. Provisional Application No. 63/632,256, filed Apr. 10, 2024, the entire contents of which are incorporated herein by reference for all purposes.

### BACKGROUND

Acceptance of oral endocrine agents as anti-cancer therapies is low due to fear of serious adverse effects. Accordingly, methods for delivering anti-cancer agents such as oral endocrine agents that maintain efficacy while avoiding undesirable side effects are needed. Endocrine agents including hydroxy tamoxifen, endoxifen, and telapristone acetate have been evaluated for local transdermal therapy (LTT) to the breast as LTT minimizes systemic drug exposure and is more acceptable to high-risk women for breast cancer prevention. However, skin permeation of the drug is low. (Z)-endoxifen (ENX), a major active metabolite of tamoxifen, has been developed as a hydroalcoholic gel with oleic acid as permeation enhancer, and was tested in a clinical trial (NCT03317405). However, large individual variation in skin permeation was seen, demonstrating that improvement in transdermal permeation is necessary for efficacy. As such, there remains a need for effective alternative methods of delivering agents such as anti-cancer therapies that maintain efficacy while reducing systemic effects.

### SUMMARY

In some aspects, provided herein are compositions comprising an endocrine agent and a transethosome. In some embodiments, the transethosome comprises ethanol, phospholipids, and one or more edge activators. In some embodiments, the endocrine agent is encapsulated within the transethosome. In some embodiments, the endocrine agent is (z)-endoxifen (ENX) or ulipristal acetate (UPA).

In some embodiments, the phospholipids comprise one or more phosphatidylcholines. For example, in some embodiments the phospholipids comprise soya phosphatidylcholine, dipalmitoyl phosphatidylcholine, distearoyl phosphatidylcholine, egg L-α-phosphatidylcholine, or a combination thereof.

In some embodiments, the one or more edge activators comprise sodium deoxycholate, sodium cholate, sodium oleate, linoleic acid, oleic acid, sorbitan stearate, sorbitan oleate, benzalkonium chloride, propylene glycol, polysorbate, or a combination thereof.

In some embodiments, a ratio of the phospholipids to the one more edge activators in the transethosome is about 99.8:02 (w/w) to about 0.2:99.8 (w/w).

In some embodiments, the transethosome comprises about 5% to about 50% ethanol. For example, in some embodiments the transethosome comprises about 5% to about 30% ethanol.

In some embodiments, the transethosome comprises water. For example, in some embodiments the transethosome comprises 50% to 95% water.

In some aspects, provided herein is a method comprising topically applying a composition provided herein (e.g. a composition comprising an endocrine agent and a transethosome comprising ethanol, phospholipids, and one or more edge activators) to a subject. In some embodiments, the subject has cancer. For example, in some embodiments the subject has breast cancer.

In some aspects, provided herein is a method of treating cancer in a subject, comprising topically applying a composition provided herein (e.g. a composition comprising an endocrine agent and a transethosome comprising ethanol, phospholipids, and one or more edge activators) to the subject. In some embodiments, the cancer is breast cancer.

### BRIEF DESCRIPTION OF THE DRAWINGS

FIG. 1A shows Z-endoxifen standard curve. FIG. 1B shows a chromatogram of 50 μg/mL Z-Endoxifen solution.

FIG. 2A shows the Zeta potential of Z-endoxifen transethosomes. FIG. 2B shows the Particle size of Z-endoxifen transethosomes.

FIG. 3 shows morphology of transethosomes as observed by transmission electron microscopy (TEM).

FIG. 4 shows a z-endoxifen standard curve after diluting in methanol.

FIG. 5 shows the drug stability curve over time. Z-ENX-TE particles showed excellent stability over the course of 30 days.

FIG. 6 shows evaluation of skin permeation of z-endoxifen encapsulated transethosomes in vivo.

FIG. 7 shows structures of endoxifen and ulipristal acetate.

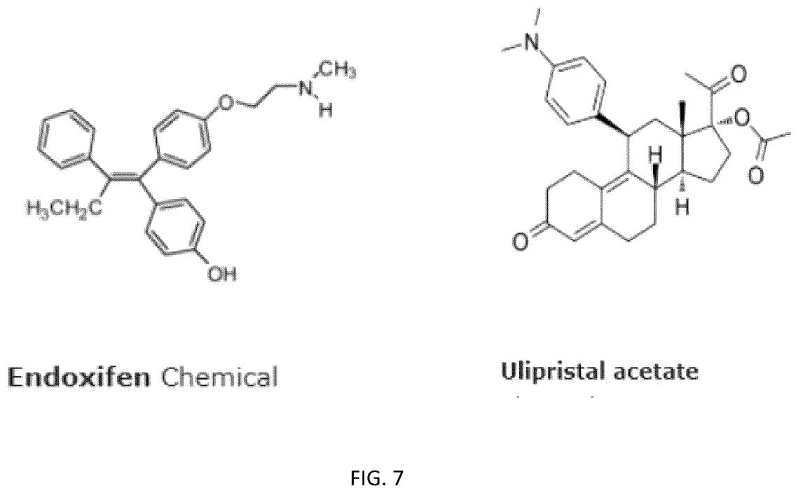

FIG. 8 shows a schedule of procedures for the experiments in Example 4.

FIG. 9 shows Transmission Electron Microscopy (TEM) images of Z-ENX-TE and vehicle-TE treated group.

FIG. 10A shows Z-ENX concentration (n=10 animals; 10 paired mammary glands) and FIG. 10B shows epithelial cell proliferation, measured by Ki67 labeling index (LI) (n=8 animals; 8 paired mammary glands) in each mammary tissue. Statistical significance was determined using the Wilcoxon matched-pairs signed rank test (two-tailed). Median difference between paired mammary glands in FIG. 10A (Z-ENX tissue concentration) was 6.9 ng/g and 7.0% for the FIG. 10B (Ki67 labeling index). FIG. 10C shows correlation between Z-ENX tissue concentration and Ki67 labeling index difference relative to the corresponding vehicle-E or DNCB-treated mammary gland. Each data point represents an individual paired comparison.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The use of the terms “a” and “an” and “the” and “at least one” and similar referents in the context of describing the invention (especially in the context of the following claims) are to be construed to cover both the singular and the plural, unless otherwise indicated herein or clearly contradicted by context.

As used herein, the modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to +10% of the indicated number. For example, “about 10%” may indicate a range of 9% to 11%, and “about 1” may mean from 0.9-1.1. Other meanings of “about” may be apparent from the context, such as rounding off; for example, “about 1” may also mean from 0.5 to 1.4.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., primates, dogs, cats, cows, horses, sheep, poultry, fish, crustaceans, etc.). In some embodiments, the subject is a human.

### DETAILED DESCRIPTION

In some aspects, provided herein are novel, skin permeable transethosomes containing clinically relevant doses of endocrine agents. Use of such transethosomes provide an alternative strategy for cancer prevention in an extremely high-risk population while avoiding first-pass hepatic metabolism and minimizing systemic drug exposure, therefore avoiding liver toxicity. Drug loaded transethosomes provide an alternative option for women who do not want to take endocrine agents orally due to concern of unwanted side effects.

In some aspects, provided herein are compositions comprising an endocrine agent and a transethosome. A “transethosome” as used herein refers to a type of highly flexible liposome. A “liposome” refers to a phospholipid bilayer formed into a substantially circular arrangement. The circular phospholipid bilayer encircles a space, refers to as a “purge space”, which can be filled/loaded with one or more desired agents. A “transethosome” refers to a type of liposome containing phospholipids and additionally containing ethanol and an edge activator (also referred to as a “permeation enhancer” or a “penetration enhancer”). Transethosomes can penetrate the skin layer by a combination of the transepidermal osmotic gradient and the effect of ethanol. Squeezing of vesicles and lipid perturbation allows the transethosome to penetrate through paracellular space, thus penetrating into deeper skin tissues and releasing the agent into systemic circulation.

In some embodiments, the transethosome comprises ethanol, phospholipids, and one or more edge activators. In some embodiments, the phospholipids are arranged in a circular phospholipid bilayer, and the ethanol and one or more edge activators are embedded within the phospholipid bilayer. In some embodiments, the endocrine agent is encapsulated within the transethosome. A transethosome containing (e.g. encapsulating) an endocrine agent is referred to herein as a “loaded transethosome” or a “drug-loaded transethosome”. The endocrine agent being “encapsulated” within the transethosome indicates that at least a portion of the endocrine agent is contained within the purge space of the transethosome. The endocrine agent being “encapsulated” within the transethosome does not necessarily indicate that all of the endocrine agent is encapsulated/contained within the purge space. For example, some of the endocrine agent can be embedded within the phospholipid bilayer. In some embodiments, the composition comprises a small amount of the endocrine agent (e.g. a trace amount) that is not encapsulated within the transethosome or embedded within the phospholipid bilayer, but exists outside of the transethosome completely (e.g. freely in solution).

The phospholipids may be natural or synthetic. As described above, the phospholipids are arranged in a substantially circular bilayer. In some embodiments, the phospholipids of the transethosome comprise one or more phosphatidylcholines. For example the in some embodiments the phospholipids comprise one or more of soya phosphatidylcholine, dipalmitoyl phosphatidylcholine, distearoyl phosphatidylcholine, egg L-α-phosphatidylcholine, or a combination thereof. In some embodiments, the transethosome comprises a plurality of different type of phospholipids. In some embodiments, the phospholipids are naturally-derived. The phospholipids may be derived from any suitable source. For example, in some embodiments the phospholipids are derived from lecithin. For example, in some embodiments the phospholipids are extracted from lecithin, including lecithin from egg yolks, marine sources, soybeans, milk, rapeseed, cottonseed, sunflower oil, etc. In some embodiments, the phospholipids are synthetic phospholipids. Synthetic phospholipids include dipalmitoyl phosphatidylcholine (DPPC), dimyristoyl phosphatidylcholine (DMPC), distearoyl phosphatidylcholine (DSPC), hydrogenated soy phosphatidylcholine, 1,2-diolcoyl-sn-glycero-3-phosphocholine, 1,2-distearoyl-sn-glycero-3-phospho-1′-rac-glycerol, and 1,2-dipalmitoyl-sn-glycero-3-phosphoglycerol.

In some embodiments, the one or more edge activators comprise a surfactant. In some embodiments, the one or more edge activators comprise a surfactant and a fatty acid or a derivative thereof. In some embodiments, the one or more edge activators comprise one or more of sodium deoxycholate, sodium cholate, sodium oleate, linoleic acid, oleic acid, sorbitan esters (e.g., sorbitan stearate (Span®60), sorbitan oleate (Span®80), sorbitan tristearate (Span®65), sorbitan monolaurate (Span®20), Span®25), benzalkonium chloride, propylene glycol, polysorbate (Tween®20, Tween®60, Tween®80), diacetyl phosphate, cetyl trimethyl ammonium bromide, dimethyl di-dodecyl ammonium bromide, or combinations thereof. In some embodiments, multiple edge activators are used. For example, in some embodiments the transethosome comprises polysorbate, sodium deoxycholate, and benzalkonium chloride.

In some embodiments, a ratio of the phospholipids to the one or more edge activators in the transethosome is about 99.8:0.2 (w/w) to about 0.2:99.8 (w/w). This ratio refers to the ratio of the phospholipids to all edge activators present in the transethosome. For example, when multiple different edge activators are present, the ratio refers to the ratio of phospholipids to all of the multiple different edge activators combined. In some embodiments, the ratio of the phospholipids to the one or more edge activators in the transethosome is about 98.8:0.2, about 98:2, about 97:3, about 96:4, about 95:5, about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, about 5:95, about 4:96, about 3:97, about 2:98, or about 0.2:99.8. In some embodiments, the ratio of the phospholipids to the one or more edge activators in the transethosome is about 3:1 to about 1:3. In some embodiments, the ratio is about 3:1 to about 1:3 or about 2:1 to about 1:2. In some embodiments, the amount of phospholipids in the transethosome is greater than the amount of edge activators in the transethosome (e.g. w/w). In some embodiments, the ratio of phospholipids to edge activators is about 2:1 to about 1.1:1. In some embodiments, the ratio is about 2:1, about 1.9:1, about 1.8:1, about 1.7:1, about 1.6:1, about 1.5:1, about 1.4:1, about 1.3:1, about 1.2:1, about 1.1:1, or about 1:1.

In some embodiments, the transethosome comprises about 5% to about 50% ethanol. In some embodiments, the transethosome comprises about 5% to about 40% ethanol. In some embodiments, the transethosome comprises about 5% to about 30% ethanol. In some embodiments, the transethosome comprises about 10% to about 20% ethanol. In some embodiments, the transethosome comprises about 5%, about 10%, about 15%, about 20%, about 25%, about 30%, about 35%, about 40%, about 45%, or about 50% ethanol.

In some embodiments, the composition further comprises water. In some embodiments, the transethosome further comprises water. In some embodiments, the transethosome comprises up to 95% water. For example, in some embodiments the transethosome comprises up to 95%, up to 90%, up to 85%, up to 80%, up to 75%, up to 70%, up to 65%, up to 60%, up to 55%, or up to 50% water. In some embodiments, the transethosome comprises 50% to 95% water, 55% to 90% water, 60% to 85% water, 65% to 80% water, or about 70% to 75% water.

In some embodiments, the endocrine agent is (z)-endoxifen (ENX). In some embodiments, the endocrine agent is ulipristal acetate (UPA). The structure of endoxifen and ulipristal acetate are shown below:

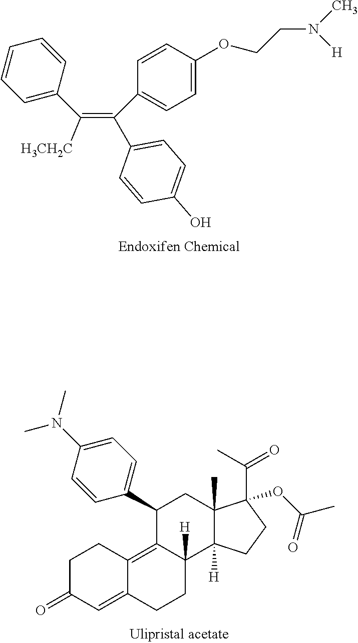

In some embodiments, the loaded transethosome contains 0.01% to 10% endocrine agent (w/w). In some embodiments, the loaded transethosome contains 0.1% to 10%, 0.2% to 9%, 0.3% to 8%, 0.4% to 7%, 0.5% to 6%, 0.6% to 5%, 0.7% to 4%, 0.8% to 3%, 0.9% to 2% or about 1% to 1.9% endocrine agent.

In some embodiments, the loaded transethosome (e.g. the transethosome containing the endocrine agent) has an average diameter of less than 500 nm. In some embodiments, the loaded transethosome has an average diameter of about 50 nm to about 500 nm. In some embodiments, the loaded transethosome has an average diameter of about 50 nm to about 300 nm. In some embodiments, the loaded transethosome has an average diameter of about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, about 300 nm. In some embodiments, the loaded transethosome has an average diameter of about 100 nm to about 200 nm. In some embodiments, the loaded transethosome has an average diameter less than 100 nm. Extrusion may be performed to achieve transethosomes of the desired size.

Various parameters of the transethosome may be modified to optimize properties of the transethosome for effective transdermal delivery of the endocrine agent contained therein to a subject. For example, the specific phospholipids and edge activator(s) may be modulated to optimize properties of the transethosome. Alternatively or in combination, the ratio of phospholipids to edge activators, and/or the amount of ethanol present in the transethosome can be modulated to optimize transethosome properties. In some embodiments, parameters are modified to optimize entrapment efficiency (EE) of the transethosome—e.g. the efficiency of entrapping the endocrine agent within the transethosome. In some embodiments, the transethosome has an entrapment efficiency of at least 80% (e.g. at least 80%, at least 85%, at least 90%, at least 95%, or at least 99%). A higher entrapment efficiency results in a composition wherein a substantial majority of the endocrine agent is encapsulated within the transethosome, and only a small amount of the endocrine agent remains free in solution (e.g. is not entrapped within the transethosome). In some embodiments, parameters are modulated to optimize the zeta potential, particle size, and particle size distribution of transethosomes, which may influence transdermal permeability and thus effect systemic delivery of the endocrine agent to the subject following application to the skin. In some embodiments, the transethosome has a negative surface charge (e.g. a negative zeta potential).

In some embodiments, the transethosome further comprises a stabilizer. The stabilizer may be any suitable moiety that imparts stability to the transethosome, prevents aggregation of transethosomes, maintains size or structure of transethosomes, and/or improves shelf life of the transethosomes. Exemplary stabilizers include, for example, hydroxypropyl-beta-cyclodextrin or cholesterol.

Transethosomes may be prepared by any suitable method. Exemplary methods for preparing transethosomes are described in the accompanying Examples, in particular Example 1.

For example, in some embodiments transethosomes are prepared by forming an ethanol phase containing the phospholipids and edge activator(s), preparing a water phase, and rapidly injecting the water phase into the ethanol phase. In some embodiments, loaded transethosomes are prepared wherein the endocrine agent (e.g. to be loaded in the transethosome, such as endoxifen or ulipristal acetate) is contained in the ethanol phase that is then injected into the water phase. In some embodiments, the ethanol phase is injected into the water phase while stirring, and at a temperature of about 60° C. to about 80° C. (e.g. about 60° C. to 80° C., about 62° C. to about 78° C., about 64° C. to about 76° C., about 66° C. to about 74° C., about 68° C. to about 72° C., or about 70° C. However, multiple methods may be used to prepare transethosomes, including the cold method, hot method, the ethanol injection-sonication method, the thin-film hydration technique, and the reverse phase evaporation method.

The cold method refers to a technique where formulation of transethosomes is performed at relatively low temperatures, thus avoiding thermal stress that can lead to degradation of the endocrine agent. The organic phase (e.g. containing the phospholipids and edge activators) is prepared by vigorously mixing the materials in ethanol. The aqueous phase is added dropwise to the organic phase and the mixture is stirred at a suitable rate. The endocrine agent will dissolve in either phase, depending on the properties of the agent and the molecule.

The hot method refers to a technique that involves application of heat during the formulation process, which enhances lipid solubility and may improve encapsulation efficiency of the drug. In this process, a colloidal dispersion of the phospholipid is formed by dispersing the phospholipid in water at a temperature of about 40 degrees C. The organic phase is added to the water phase and stirred continuously to form a suspension. The endocrine agent can be dissolved in either water or ethanol, depending on the properties of the agent.

The ethanol injection sonication method involves dissolving phospholipids, the edge activators, and the endocrine agent in ethanol. This organic phase is then injected into the aqueous phase through a syringe, and the mixture is homogenized.

The thin-film hydration technique involves creating a slender lipid layer on a rotary evaporator flask, which lipid film is hydrated by introducing water or an aqueous buffer. Phospholipids, edge activators, and the endocrine agent are dissolved in the organic phase in a round bottom flask and homogenized. Organic solvents are then slowly removed to form the thin lipid film. The dried lipid film is then diluted with an aqueous solution of ethanol or a suitable ethanol buffer and then allowed to swell into vesicles.

For any method of producing transethosomes, extrusion may be performed to achieve the desired particle size. The specific method for transecthosome production used may depend on the components of the transethosome (e.g. the specific phospholipids, the specific edge activators), the amounts thereof, the desired size of the transethosome to be produced, and the hydrophobicity/hydrophilicity of the endocrine agent to be encapsulated.

In some aspects, provided herein are methods of use of the compositions provided herein. In some embodiments, provided herein are methods of treating cancer in a subject, comprising providing to the subject a composition comprising a transethosome encapsulating an endocrine agent (e.g. a loaded transethosome) as described herein. In some embodiments, provided herein is a method comprising topically applying a composition described herein to a subject. In some embodiments, the subject has subject has cancer. In some embodiments, the subject has breast cancer. In some embodiments, topical application of the composition achieves transdermal delivery of the endocrine agent (e.g. the endocrine agent encapsulated within the transethosome) to the subject, thereby effectively treating cancer in the subject. In some embodiments, transdermal delivery of the endocrine agent exerts systemic effects while avoiding unwanted side effects otherwise associated with oral or parenteral administration of the endocrine agent to the subject.

In some embodiments, the composition is topically applied to an area proximal to the cancer in the subject. For example, in subjects having breast cancer the composition is applied to the breast of the subject, thereby achieving transdermal delivery of the endocrine agent to the cancerous breast tissue. In some embodiments, the composition is topically applied daily. In some embodiments, the composition is topically applied more than once per day (e.g. 2 times per day, 3 days per day, 4 times per day, etc.). In some embodiments, the composition is topically applied more than once per day, once per day, once every 2 days, once every 3 days, once every 4 days, once every 5 days, once every 6 days, weekly, once every 8 days, once every 9 days, once every 10 days, once every 11 days, once every 12 days, once every 13 days, once every 14 days, every 3 weeks, every 4 weeks, every 5 weeks, every 6 weeks, every 7 weeks, every 8 weeks, or less frequently. In some embodiments, the composition is applied 1 to 7 times per week (e.g. 1-7 times, 2-6 times, 3-5 times).

In some embodiments, dose of the endocrine agent provided to the subject in the composition comprising the transethosome loaded with the endocrine agent is lower than the dose that would otherwise be required for effective skin permeation following transdermal application of the equivalent non-transethosome encapsulated endocrine agent. In some embodiments, the composition is topically applied at a reduced frequency compared to the frequency of administration that would be otherwise required for the equivalent non-transethosome encapsulated endocrine agent. Accordingly, the loaded transethosomes and compositions provided herein achieve skin penetration of the endocrine agent, a relatively high concentration of the agent in the target tissue (e.g. in the mammary tissue), while avoiding significant systemic dissemination and unwanted side effects.

In some embodiments, an amount of the composition is topically applied such that the dose of the endocrine agent provided to the subject (e.g. provided to the breast tissue) is about 1 mg to about 10 mg. In some embodiments, an amount of the composition is topically applied to the breast such that the dose of the endocrine agent provided to the subject (e.g. to the breast tissue) is about 1 mg, about 2 mg, about 3 mg, about 4 mg, about 5 mg, about 6 mg, about 7 mg, about 8 mg, about 9 mg, or about 10 mg.

### Example 1

Phospholipids, surfactants and ethanol were used to prepare Z-Endoxifen (ENX)-encapsulated transethosomes (Z-ENX-TE).

### Preparation Steps:

### Ethanol Phase

375.0 mg of egg yolk lecithin, 150.0 mg of Tween-80, 79.5 mg of sodium deoxycholate, 3.75 mg of 80% benzalkonium chloride, and 30.4 mg of Z-Endoxifen were precisely weighed. 3 mL of ethanol (anhydrous) was added and reagents were dissolved ultrasonically in a water bath.

### Water Phase

9 mL of pure water was precisely measured and heated to 70° C. in a water bath.

### Mix

The water phase was rapidly injected into the ethanol phase under high-speed stirring with a magnetic stirrer. The reaction temperature was set to 70° C. The reaction was sealed for 30 min until the solution was clear, no suspended particles were seen with the naked eye, and there was a clear light path under the laser.

### Characterization:

After cooling the transethosomes to room temperature, the remaining volume was 10 ml. The total drug concentration was determined by HPLC using 20 μL demulsified samples. 200 μL samples were collected into a 30 kDa ultrafiltration tube, added 1 mL of 25% ethanol, ultrafiltrated at 5000 rpm, and the drug concentration of subnatant was determined by HPLC.

### Storage:

Transethosomes were diluted to 15 mL and stored at room temperature to mature overnight. There was no sedimentation, no delamination, and no suspended particles. There was a clear optical path under the laser. There were no significant changes in the hydrodynamic size and zeta potential test results. Particle properties were stable. In order to maintain the osmotic pressure inside and outside the transethosomes and prevent drug leakage from the inside of the transethosomes, a trace amount of free drug was retained outside the transethosomes.

### Encapsulation Efficiency:

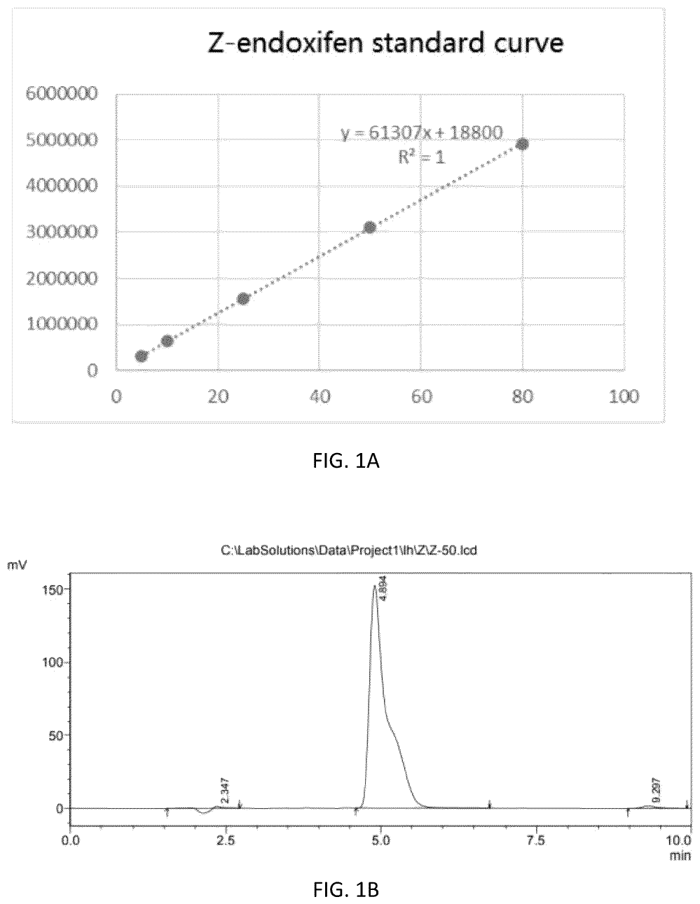

Encapsulation efficiency was evaluated by HPLC. HPLC was performed using a WondaSil® C-18-WR column (4.6×150 mm, 5 μm pore size). The mobile phase included an A phase and a B phase at a ratio (A:B) of 30:70. The A phase contained 10 mM ammonium formate in water: 10 mM ammonium formate in methanol. The B phase contained 10 mM ammonium formate in methanol at a ratio of 40:60. Samples were prepared as follows. The solvent was prepared by mixing 10 mM ammonium formate in a solution containing water and acetonitrile at a ratio of 1:1 (v/v). Z-Endoxifen was diluted in the solvent at a concentration gradient of 5, 10, 25, 50, and 80 μg/mL. Samples were injected into the column at a volume of 20 μL and were passed through the column at a flow rate of 0.8 mL/min at a temperate of 30° C. The standard curve is shown in FIG. 1A. A Chromatogram is shown in FIG. 1B. HPLC results are shown in Table 1. The detection wavelength was 243 nm.

TABLE 1
HPLC Results

| Compound | Z-Endoxifen |
|---|---|
| Dilution Factor | 100 |
| Peak Area | 1879397.67 |
| Drug concentration after dilution (μg/ml) | 30.35 |
| Total Drug Concentration (μg/ml) | 3035 |
| Total amount of drugs in the system | 30.35 |
| Subnatant dilution factor | 6 |
| Peak area of subnatant | 2346744.67 |
| Subnatant drug concentration after dilution (μg/ml) | 37.95 |
| Total drug concentration in subnatant (μg/ml) | 227.7 |
| Drug concentration in liposomes (μg/ml) | 2807.3 |
| Drug loading amount (mg) | 28.07 |
| Encapsulation efficiency (%) | 92.34% |
| Drug loading (%) | 4.77% |

The following equations were used in Table 1:

$$Drug concentration in transethosomes = total drug concentration - subnatant drug concentration$$

$$Drug loading amount = Drug concentration in transethosomes \times total volume of the system$$

$$Encapsulation Efficiency ( \% ) = Encapsulated drug concentration / Total drug concentration \times 100 \%$$

$$Drug loading = Encapsulated drug / ( Encapsulated drug + Inactive Ingredients ) \times 100 \%$$

### Zeta Potential & Particle Size

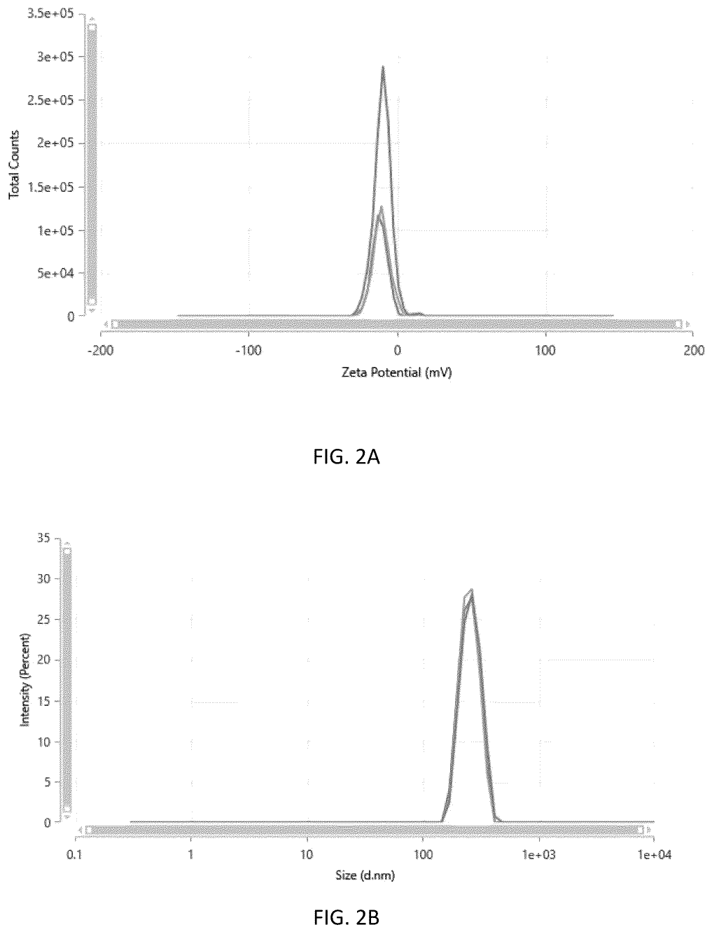

The zeta potential & particle size were characterized by the Nano Particle Size and Zeta Potential Analyzer. Results are shown in FIG. 2A and FIG. 2B, and Table 2.

TABLE 2
Zeta potential and Particle size of Z-endoxifen transethosomes

| Name | Mean | Minimum | Maximum |
|---|---|---|---|
| Intensity(nm) | 260.2 | 256.4 | 266 |
| Number(nm) | 214.9 | 212.1 | 218 |
| PDI | 0.1411 | 0.114 | 0.1744 |
| Zeta Potential(mV) | −10.5 | −11.6 | −9.707 |

### Morphology

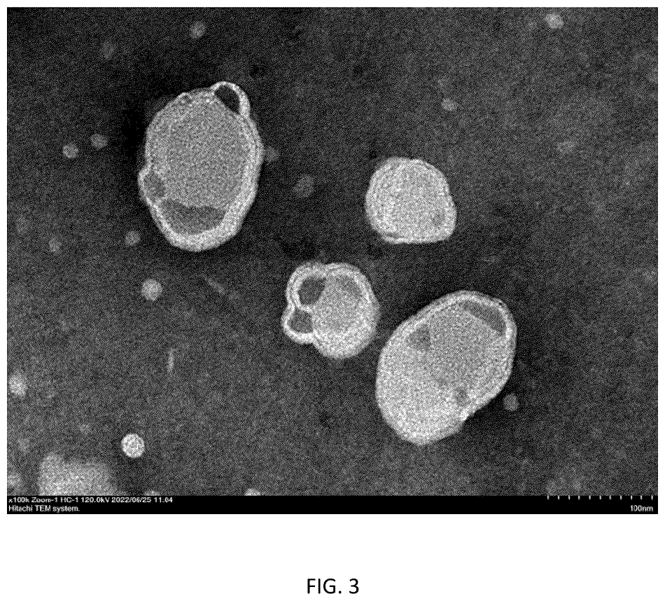

The morphology of the transethosomes was observed by Transmission electron microscopy (TEM, FIG. 3). TEM showed that the particle size of the transethosomes was about 100-200 nm, and there were scattered micelles in the solution system.

### Stability Test (HPLC)

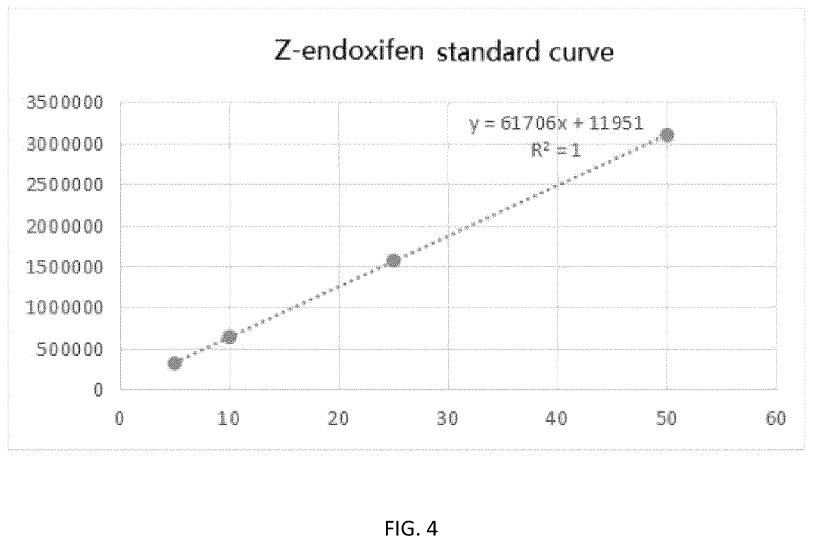

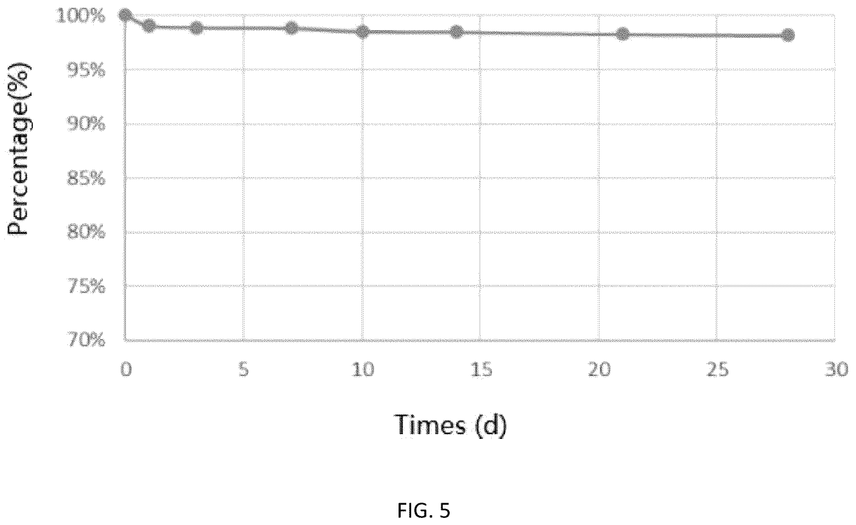

5 mL of drug-loaded transethosomes suspension was placed in a centrifuge tube, and the suspension was shaken away from light at 100 rpm under 37° C. Samples were taken at 0, 1, 3, 7, 10, 14, 21 and 28 days, and 1 mL of free drug was obtained by ultrafiltration with 10K ultrafiltration tube. The sample was supplemented with 1 mL of 25% ethanol solution to restore the original volume, and the sample was incubated on the shaker. The concentration of the obtained subnatant sample containing free drug was detected by HPLC after dilution of methanol for 2 times. The curve was drawn with time according to the standard curve 2 in FIG. 4. Data are listed in Table 4 and summarized in Table 3. Particles were stable over the course of 1 month (FIG. 5).

TABLE 3
HPLC Test Data Summary

| | |
|---|---|
| Liposome concentration (μg/ml) | 3035 |
| Encapsulation Efficiency (%) | 92.34 |
| Test volumn (mL) | 5 |
| Total drug (μg) | 15175 |
| Free drug (μg) | 1162.405 |
| Encapsulated drug (μg) | 14012.595 |

TABLE 4
HPLC Test Data

| time (d) | 0 | 1 | 3 | 7 | 10 | 14 | 21 | 28 |
|---|---|---|---|---|---|---|---|---|
| 1 | 0 | 885022 | 894723 | 804577 | 911489 | 783481 | 804087 | 745754 |
| 2 | 0 | 888298 | 871830 | 741982 | 881165 | 777902 | 794066 | 754573 |
| 3 | 0 | 890159 | 878262 | 784665 | 893610 | 765166 | 821264 | 733881 |
| Peak area | 0 | 887826.33 | 881605 | 777074.67 | 895421.33 | 775516.33 | 806472.33 | 744736 |
| Concentration | 0 | 14.17 | 14.07 | 12.37 | 14.3 | 12.34 | 12.85 | 11.84 |
| (μg/ml) | | | | | | | | |
| Dilution time | 0 | 28.35 | 28.15 | 24.74 | 28.6 | 24.69 | 25.7 | 23.68 |
| Cumulative drug | 0 | 141.75 | 169.1 | 180.2 | 224.24 | 233.29 | 263.03 | 278.63 |
| release (μg) | | | | | | | | |
| Remaining drug | 100% | 99.07% | 98.89% | 98.81% | 98.52% | 98.46% | 98.27% | 98.16% |
| quantity (μg) | | | | | | | | |

In sum, Z-Endoxifen was encapsulated in alcohol-containing transethosomes, and related tests were performed. The drug concentration in the Z-Endoxifen transethosomes was 1.871 mg/mL, the drug loading was 4.77%, and the encapsulation efficiency was 92.34%; the particle size and TEM showed that the transethosomes had good dispersibility, and there were scattered micelles in the system; Zeta potential detection indicated that the particle surface was negatively charged.

### Permeation Study

Skin samples can be used to evaluate permeation of the particles. The skin sample can be adjusted on a Franz diffusion cell with drug solution in the donor chamber, and receiver fluids (such PBS or saline) in receptor chamber. The temperature of the cell can be maintained constant. After 24 h, the receiver fluid can be selected and extracted by organic solvents. The amount of permeated drug can be evaluated by LC-MS to calculate cumulative permeation quantity (Q).

### Example 2

### Synthesis and Characterization of Drug Loaded Transethosomes

Multiple candidate formulations were designed comprising of soya phosphatidylcholine, sodium deoxycholate, Tween 80, Span 60, water, and ethanol. Synthesis of UPA and ENX-loaded transethosomes can be performed characterized by various properties.

Characterization of drug-loaded transethosomes can be performed as follows:
1) Drug Loading (DL) and Entrapment Efficiency (EE). An ultrafiltration technique is used to remove un-entrapped drug. The drug loaded in transethosomes are extracted out by organic solvents (such as DMSO) and then measured by HPLC. The DL and EE of resulting transethosomes are calculated according to the following equations: DL=mass of entrapped drug/(mass of entrapped drug+transethosomes)×100% and EE (%)=Entrapped drug/Total drug×100%. In some embodiments, it is desirable to maximize entrapment efficiency.
2) Size measurement: The average diameter of synthesized transethosomes can be measured by dynamic light scattering (e.g. using a laser particle size analyzer). In some embodiments, it is desirable to minimize particle size.
3) Determination of surface charge (zeta potential, ZP): Zeta potential is used as an indicator of stability of colloidal dispersions. Samples are diluted with the media used for their preparations and analyzed by zeta potential meter at room temperature. In some embodiments, it is desirable to maximize zeta potential.
4) Size distribution (PDI) and morphology evaluation: The polydispersity index (PDI) is used as an indicator of heterogeneity of particle size. Synthesized transethosomes are observed by Transmission Electron Microscopy (TEM). TEM is one of the most efficient, high-resolution techniques available for studying structural details, size distribution (PDI), and morphology of nanoparticles. One drop of dispersion can be negatively stained on a copper grid. The excess suspension can be immediately adsorbed using filter paper. After drying, the grid can be viewed using TEM. In order to determine the optimum formula, the desirability function that predicts the optimum levels of investigated factors can be calculated. The criterion set for selecting the optimum formula is a combination of the minimum values for PS and PDI and the maximum values for EE % and ZP (as absolute value). In some embodiments, it is desirable to minimize PDI.

A matrix of contributing factors (independent variables) such as surfactant types (Tween80, Span60) and ratio of phospholipid to surfactant (w/w), and the measured responses (dependent variables) such as EE %, PS, PDI, zeta potential (ZP) can be created to determine the optimal formulation. Regression analysis for the responses of PS, PDI and ZP for all the combinations investigated can be performed to select the optimum formula. The optimal transethosome formula may have an EE >80%, ZP >20, PS <30 nm, and PDI <0.1 for each drug (ENX and UPA). These synthesized transethosome can be tested to determine drug bioavailability (Q24 hrs) by skin permeation. In some embodiments, it is desirable to maximize drug bioavailability.

### Evaluation of Skin Permeation In Vivo:

Drug bioavailability can be determined by measuring plasma concentration of drugs delivered across skin using transethosome. Transethosome-based delivery can be compared to suitable reference groups, such as oral administration of the drug (e.g. oral administration of UPA or oral administration of ENX).

Any suitable model may be used to evaluate drug bioavailability. In some embodiments, a rodent model is used. For example, mice can be anesthetized, depilation cream (Nair) applied to remove hair on their upper dorsal area (>2×2 cm2), and rinsed with water. Animals can be randomized into suitable treatment groups to evaluate drug bioavailability of the transethosome formulation. For example, treatment groups may be developed to compare drug bioavailability of the transethosome after topical administration compared to availability of the equivalent oral agent. For example, treatment groups can include: 1) ENX hydroalcoholic gel used in the clinical trial (NCT03317405). 2) ENX-transethosome, 3) UPA-transcthosome, 4) UPA HED 5 mg P.O. Animals can anesthetized and depilated skin area rinsed with sterile water and damp-dried ready for transdermal drug application. The same dose of ENX hydroalcoholic gel, ENX-transethosome, and UPA-transethosome formula (e.g. 25 μg/day for mouse=HED 5 mg/day for each drug) should be applied to cover the desired skin area and the formula gently rubbed with circular motion using applicator until it damp-dried.

Single dosing and repeated dosing (3 and 6 dosing) experiments can be performed using a suitable number of animals (e.g. mice) per time point per treatment group. The mice can be sacrificed to collect blood samples (EDTA tubes for plasma). For example, blood samples can be collected by heart puncture at three time points (day 2, day 4, and day 7). Plasma drug concentration of ENX, UPA and their metabolites such as 4′-OH and desmethyl metabolites produced by hepatic metabolism can be determined.

### Example 3

Synthesis, characterization & in vivo transdermal delivery of (Z)-endoxifen loaded transethosome ((Z-ENX-TE)): (Z)-endoxifen was encapsulated in alcohol-containing transethosomes (transethosome) as described in Example 1. The formulation consists of lecithin 1.25%; sodium deoxycholate 2.65%, polysorbate 80 0.5%, benzalkonium chloride 0.01%, ethanol 10% & (Z)-endoxifen 0.096% (w/v). The product was diluted in water for optimal dispersion with a clear optical path under the laser. There was no sedimentation, delamination, nor suspended particles. Particles had an average size of 100-200 nm, as observed by TEM. The drug encapsulation efficiency was 92.3%. The (Z)-endoxifen concentration was 1.87 mg/mL in transethosome solution with a good dispersibility (mean PDI of 0.14) and negatively charged surface (zeta potential of −10.5 mV). Particle properties were stable over 1 month.

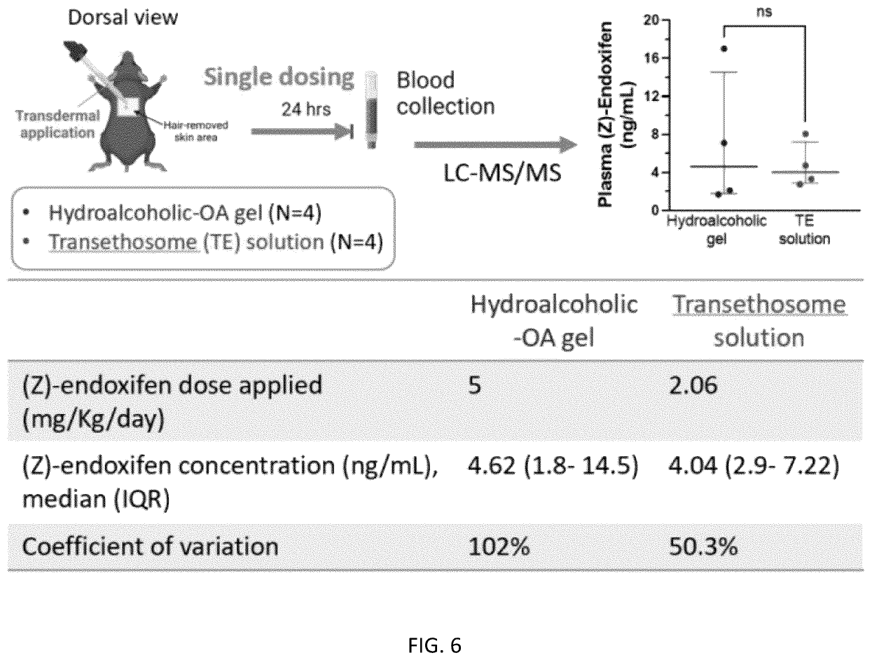

Transdermal delivery of (Z)-endoxifen-loaded transethosome was compared to a hydroalcoholic-oleic acid (OA) gel (E/Z) endoxifen racemic mixture which demonstrated poor transdermal drug delivery in a phase I trial (NCT03317405). A single dosing experiment of transdermal application was designed to measure fast drug delivery in mice. Experiments were performed in accordance with the schematic shown in FIG. 6. Z-ENX-TE was applied transdermally in a single dose, blood was collected 24 hours later and LC-MS/MS was performed to evaluate permeability. Despite a 2.4-fold lower dose of (Z)-endoxifen in the Z-ENX-TE (2.06 mg/kg) compared to the dose applied in the hydroalcoholic OA gel (5 mg/kg), the transethosome delivered (Z)-endoxifen equivalent to (E/Z) endoxifen hydroalcoholic-OA gel with a coefficient of variation for delivery was half of that endoxifen hydroalcoholic-OA gel. As such, the results demonstrate that transdermal application of Z-ENX-TE effectively penetrates the skin to deliver the active agent to the subject while requiring a lower dose of endoxifen compared to the equivalent dose currently used in hydroalcoholic OA gel.

### Example 4

### Dermal Safety and Mammary Tissue Delivery of (Z)-Endoxifen-Loaded Transethosomes in Guinea Pigs

Guinea pigs possess a pair of mammary glands similar to those in humans and are considered a regulatory dermatology model for skin sensitization testing using the Draize scoring method. This study aimed to evaluate the following: 1) local tolerance and dermal safety of (Z)-endoxifen-loaded transethosomes (Z-ENX-TE) by monitoring for any visible reactions (e.g., erythema, edema) at the site of application, 2) drug concentrations in mammary tissue and blood samples, and 3) cell proliferation in mammary tissue, assessed by Ki67 immunohistochemistry.

To optimize relevance, the skin area overlying the mammary glands—characterized by lower hair density—was selected for application. The peri-areolar skin around the nipple-areolar complex was treated with either Z-ENX-TE, vehicle-TE (drug-free transethosomes), or a positive control, dinitrochlorobenzene (DNCB), which is known to induce erythema and edema upon topical application. Z-ENX-TE was applied at a human equivalent dose (HED) of 5 mg/day (based on a 60 kg human), administered to guinea pigs three times per week for four weeks. Blood samples were collected twice weekly for plasma drug concentration analysis, and dermal irritation at application sites was scored five days per week. At study termination, mammary gland tissues and heart blood were collected to quantify Z-ENX levels. Mammary glands were also processed into formalin-fixed, paraffin-embedded (FFPE) blocks for Ki67 immunohistochemical analysis. Drug exposure following transdermal administration of ENX-TE in blood and mammary gland tissue was measured by liquid chromatography-tandem mass spectrometry (LC-MS/MS).

The results herein demonstrate that Z-ENX-TE and vehicle-TE do not elicit dermal irritation compared to the positive control, while Z-ENX-TE results in significantly higher drug concentrations in the treated mammary gland compared to the contralateral gland (vehicle-TE or DNCB-treated), with minimal systemic exposure over the 4-week period.

### Methods

### Synthesis of Z-ENX-TE

(Z)-endoxifen (ENX)-loaded transethosomes (Z-ENX-TE) were prepared for transdermal delivery in guinea pigs using the methods described herein.

### Local Tolerance and Dermal Toxicity Test

A local tolerance study was conducted as a 30-day repeat-dose study in Hartley albino guinea pigs. The study tested one intermittent dosing regimen: ENX at 0.383 mg/kg/day, corresponding to the human equivalent dose (HED) of 5 mg/day, administered every other day (three times per week) over a 4-week period.

### Animals

15 female Hartley albino guinea pigs (Crl: HA Strain Code: 051, Charles River Laboratories, Inc) were used in this study. Animals were approximately 6 weeks old on arrival and healthy, free of external parasites and having unblemished hair coats. Animals weighed 300-350 g one day after arrival. Animals were acclimated for 2 weeks until the initiation of dosing. 8 weeks old female guinea pigs weighed on average 500±80 g (mean±standard deviation). Ear tag was placed for animal identification.

### Treatments

Topical treatment were prepared as follows:

A DNCB solution (0.05% (w/v) 1-chloro-2,4-dinitrochlorobenzene (DNCB)) was prepared as a positive control. DNCB is a known skin irritant and contact allergen. DNCB was formulated as a 0.05% (w/v) solution in 95% ethanol (200 proof, anhydrous, >99.5%) the day prior to dosing the positive control group. The solution was aliquoted for fresh use per each doing. Application volume was 70 uL.

Drug free transethosome (vehicle-TE solution) was prepared using egg yolk lecithin, Tween-80, sodium deoxycholate, benzalkonium chloride, and ethanol in water. The application volume was 34 uL.

The Z-ENX-TE solution was prepared using the same constituents as the vehicle-TE solution and loaded with Z-ENZ at a human equivalent dose of 5 mg/day. The application volume was 34 μL.

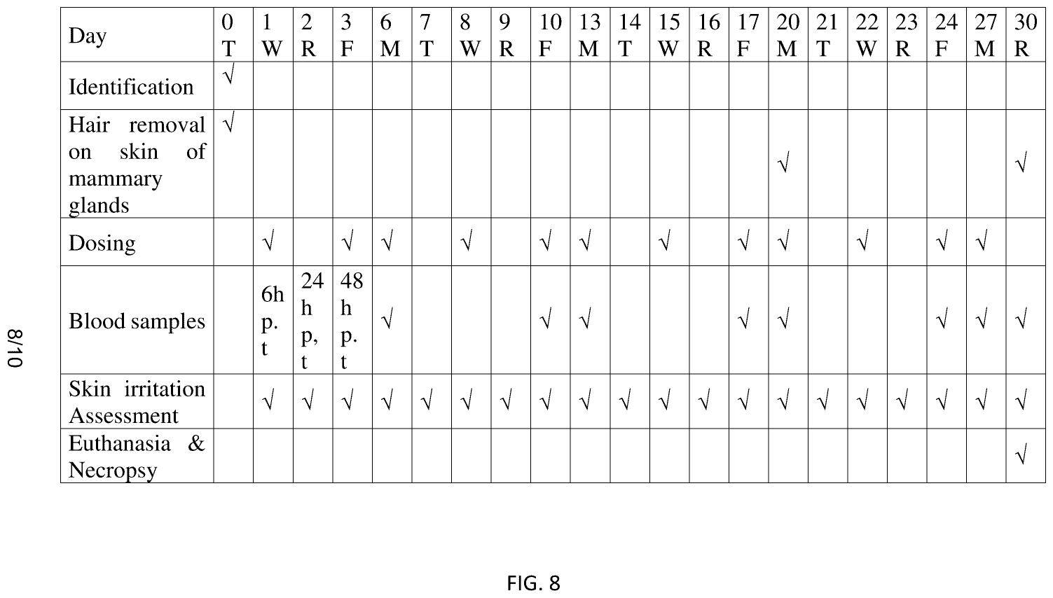

The following animal groups were used (5 animals per group). A dosing frequency of 3 doses per week was used for all groups.
Group 1: Z-ENX-TE applied to one side, vehicle-TE applied to other side.
Group 2: Z-ENX-TE applied to one side, DNCB applied to other side.
Group 3: DNCB applied to one side, vehicle-TE applied to other side.
   All procedures and clinical evaluations were conducted according to the planned dosing schedule shown in FIG. 8.

### Skin Preparation

The day prior to the first dosing, the abdominal fur of each guinea pig was removed with hair clippers following with application of depilatory cream (Nair™ Sensitive Formula) to the clipped abdomen. The depilatory cream was wiped off with gauze, washed off with warm water within 10 minutes of application, then the abdomen was gently dry with a soft towel. The guinea pigs were rested for at least 4 hours before administering the test agents.

### Animal Treatment

A 19-20 mm diameter ring was placed around each nipple to guide topical application. As shown in Table 5, each treatment was applied inside the ring (avoiding nipple) and gently massaged 10 times for adsorption with the Q-tips covered with surgical glove material. Once dried, each treated skin area was covered with a Tegaderm to prevent cross contamination between the two application sites. Animals were collar worn and individually housed to prevent a chance of drug ingestion. Tegaderm were worn for 6 hours until monitoring animals for dermal irritation. Prior to every new dosing, the application site was gently wiped with a clean tissue pad soaked in nuke warm water to remove (as applicable) any residual dosing material and gently pad dry with a sterile gauze or paper towel.

### Skin Irritation Evaluation

Dermal irritation at the topical application site was assessed for edema and erythema and/or eschar formation using the Draize scoring method (Charmeau-Genevois C, Sarang S, Perea M, Eadsforth C, Austin T, Thomas P. A simplified index to quantify the irritation/corrosion potential of chemicals-Part I: Skin. Regul Toxicol Pharmacol. 2021; 123:104922), which employs a 0-4 scale (see Table 5). Two trained staff members, a veterinarian and an animal health technician performed the evaluations. Assessments were conducted: Pre-dose and approximately 6 hours post-dose on Study Day 1; Once daily on weekdays at approximately the same time (˜6 hours post-dose on Day 1) from Days 2 through 27; At necropsy on Day 30. Each treatment site was evaluated for signs of dermal irritation, including edema, erythema, and/or eschar (defined as superficial crust or scab) formation.

TABLE 5
Evaluation of Skin Reactions

| Skin reaction | Clinical signs | Value |
|---|---|---|
| Erythema and eschar | No erythema | 0 |
| (crust or scab) | Very slight erythema (barely perceptible) | 1 |
| formation | Well-defined erythema | 2 |
| | Moderate to severe erythema | 3 |
| | Severe erythema to slight eschar formation | 4 |
| Edema formation | No edema (barely perceptible) | 0 |
| | Very slight edema (raised edges of area well-defined) | 1 |
| | Slight edema | 2 |
| | Moderate edema | 3 |
| | Severe edema (raised more than 1 mm and extending | 4 |
| | beyond the area of exposure) | |

### Blood Collection

Approximately 0.2 mL of blood was drawn from the lateral saphenous or tarsal vein of guinea pigs and collected into BD K2-EDTA tubes for plasma isolation. Tubes were gently inverted several times to ensure proper mixing with the anticoagulant, then centrifuged at 12×g for 15 minutes at 4° C. The resulting plasma supernatant was carefully transferred into cryogenic tubes and stored at −80° C. until further analysis.

### Euthanasia

Animals were asphyxiated using CO₂ followed by decapitation. Using a non-precharged chamber, CO₂ was dispensed from a commercial cylinder with fixed pressure regulator and inline restrictor controlling gas flow within 30%-70% of the chamber volume per minute to comply with 2020 AVMA Guidelines.

### Necropsy

The final blood sample was collected via cardiac puncture and processed. Abdominal skin hair was removed using Nair depilatory cream, followed by cleansing with 60% alcohol pads to eliminate any residual drug from the skin surface prior to tissue collection. A Y-shaped skin incision was made to avoid the genital area, and the skin was reflected to expose the mammary glands. To minimize cross-contamination of tissue specimens due to potential drug carryover, each mammary gland was excised by two separate personnel using new, sterile surgical tool kits for each specimen. Approximately 20 grams of mammary tissue (mastectomy specimen) were collected per gland. The tissue was divided as follows: The portion distal to the nipple was snap frozen for drug concentration analysis. The portion proximal to the nipple was fixed in 10% neutral-buffered formalin for 48 hours, then processed to prepare formalin-fixed paraffin-embedded (FFPE) blocks for histopathological evaluation. In addition, the full-thickness skin from the treatment area was exercised, fixed in 10% neutral-buffered formalin for 48 hours, and processed into paraffin blocks for future histopathological analysis.

### Drug Quantitation

Mammary tissue and plasma samples were analyzed by LC-MS/MS method.

### Results

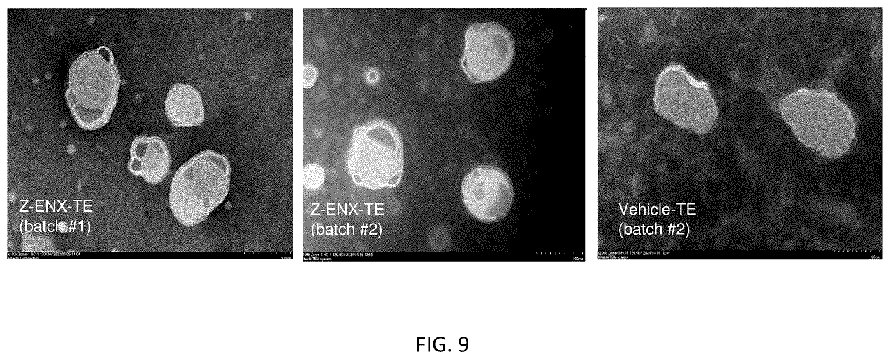

The Z-ENX concentration in the Z-ENX-TE was 3.315 mg/mL, and the encapsulation efficiency was 97.5%; the particle size and TEM showed good dispersibility and there were scattered micelles in the system. The physical characteristics of Z-ENX-TE in batches prepared at different time points were compared (Batch #1, Batch #2). Material stability over time of each batch is summarized in Table 6. The morphologies of the empty (vehicle) and Z-ENX-TE were observed by transmission electron microscopy (FIG. 9). Overall, the nano-vesicle size of batch 2 was slightly smaller than batch 1. In addition, drug encapsulation efficiency and TE vesicle stability were slightly higher than batch 1. These variations can be accounted for by normal batch variation.

TABLE 6
Material stability of (Z)-endoxifen-
loaded transethosome (Z-ENX-TE)

| | | | | | | Remaining |
|---|---|---|---|---|---|---|
| | | Size | | Zeta | | drug in |
| Batch | Month | (nm) | PDI | (mv) | EE | TE % |
| #1 | 0 | 260.2 | 0.14 | −10.5 | 92.3% | 100% |
| | 1 | | | | 90.6% | 98.2% |
| | 10 | | | | 88.0% | 95.3% |
| #2 | 0 | 195.9 | 0.18 | 0.76 | 97.5% | 100% |
| | 1 | 225.4 | 0.13 | −16.5 | 97.0% | 99.4% |
| | 6 | 232.8 | 0.29 | −10.5 | 96.7% | 99.2% |

The local tolerance and dermal sensitization potential of Z-ENX-TE and vehicle-TE were evaluated in Hartley albino guinea pigs. A positive control group, consisting of 10 guinea pigs, received 0.05% 1-chloro-2,4-dinitrobenzene (DNCB) in 95% ethanol. A dermal sensitization response was considered positive if two or more reactions with an erythema score of 1 or greater occurred following multiple doses. Clear evidence of sensitization was observed in the DNCB-treated group, confirming the validity of the study. After the 7th DNCB dose (during the third week of treatment), erythema scored as 2 (well-defined) was seen in 3 of 10 animals, while the remaining 7 animals showed erythema scored as 1. In contrast, no animals treated with Z-ENX-TE or vehicle-TE exhibited erythema scores of 1 or greater lasting more than one day. These findings indicate that Z-ENX-TE and its vehicle were well tolerated when applied every other day to the skin over the mammary gland for up to 30 days. It is possible that none of the components in the vehicle-TE formulation are irritating to human skin.

### Drug Concentration and Epithelial Cell Proliferation (Ki67 LI)

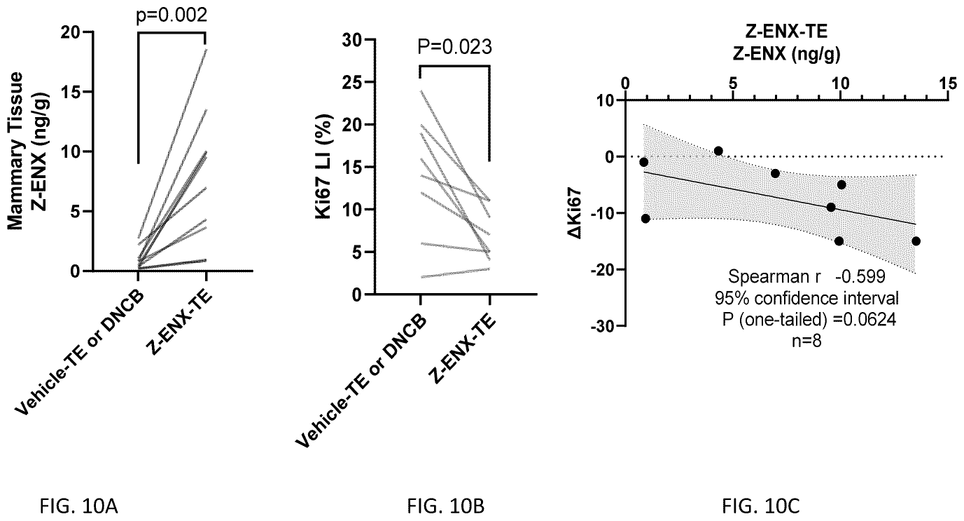

The median Z-ENX concentration in mammary tissue was approximately 13 times higher at the Z-ENX-TE-treated site—8.27 ng/g (IQR: 2.97-10.9 ng/g)—compared to 0.64 ng/g (IQR: 0.26-1.34 ng/g) at the vehicle-TE or DNCB-treated site (p=0.002) (FIG. 10A). The Z-ENX concentration achieved in mammary tissue via transdermal Z-ENX-TE (˜10 ng/g) is comparable to that observed following oral administration of 20 mg tamoxifen in women. Transdermal Z-ENX-TE led to a significant reduction (median 7.0%) in epithelial cell proliferation, as measured by Ki67 labeling index (LI), relative to the vehicle-TE or DNCB-treated mammary gland (p=0.023) (FIG. 10B). A trend indicating that higher Z-ENX concentrations in mammary tissue were associated with reduced epithelial cell proliferation was observed, although this association did not reach statistical significance (FIG. 10C). The median plasma concentration of Z-ENX across 10 animals over 30 days was 2.87 ng/ml (IQR: 1.97-4.56 ng/mL). The tissue-to-blood ratio of Z-ENX concentration was 2.9, indicating high drug retention in mammary tissue with relatively low systemic exposure. Taken together, the results herein demonstrate effective local transdermal therapy for the breast using Z-ENX-TE.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

What is claimed is:

1. A composition comprising:
a) an endocrine agent; and
b) a transethosome comprising ethanol, phospholipids, and one or more edge activators, wherein the endocrine agent is encapsulated within the transethosome.

2. The composition of claim 1, wherein the endocrine agent is (z)-endoxifen (ENX) or ulipristal acetate (UPA).

3. The composition of claim 1, wherein the phospholipids comprise one or more phosphatidylcholines.

4. The composition of claim 3, wherein the phospholipids comprise soya phosphatidylcholine, dipalmitoyl phosphatidylcholine, distearoyl phosphatidylcholine, egg L-α-phosphatidylcholine, or a combination thereof.

5. The composition of claim 1, wherein the one or more edge activators comprise sodium deoxycholate, sodium cholate, sodium oleate, linoleic acid, oleic acid, sorbitan stearate, sorbitan oleate, benzalkonium chloride, propylene glycol, polysorbate, or a combination thereof.

6. The composition of claim 1, wherein a ratio of the phospholipids to the one more edge activators in the transethosome is about 99.8:02 (w/w) to about 0.2:99.8 (w/w).

7. The composition of claim 6, wherein the ratio is about 3:1 to about 1:3.

8. The composition of claim 7, wherein the ratio is about 2:1 to about 1:1.

9. The composition of claim 1, wherein the transethosome comprises about 5% to about 50% ethanol.

10. The composition of claim 9, wherein the transethosome comprises about 10% to about 30% ethanol.

11. The composition of claim 1, wherein the transethosome further comprises water.

12. The composition of claim 11, wherein the transethosome comprises about 50% to about 95% water.

13. The composition of claim 11, wherein the transethosome comprises about 70% to about 80% water.

14. The composition of claim 1, wherein the transethosome contains 0.01% to 10% endocrine agent (w/w).

15. A method comprising topically applying the composition of claim 1 to a subject.

16. The method of claim 15, wherein the subject has cancer.

17. The method of claim 16, wherein the subject has breast cancer.

18. A method of treating cancer in a subject, the method comprising topically applying the composition of claim 1 to the subject.

19. The method of claim 18, wherein the cancer is breast cancer and wherein the composition of claim 1 is topically applied to the breast of the subject.

20. The method of claim 19, wherein the composition is topically applied in an amount such that a dose of the endocrine agent provided to the breast tissue of the subject is about 1 mg to about 10 mg.
